(19) United States
(12) Patent Application Publication
(10) Pub. No.: US 2017/0258924 A1
(43) Pub. Date: Sep. 14, 2017

(54) Ester of a Phospholipid With Conjugated Linoleic Acid for the Treatment of Psychiatric Disorders With Neuroinflammatory and Neurodegenerative Basis

(71) Applicant: UNIVERSITA' DEGLI STUDI DI CAGLIARI, Cagliari (IT)

(72) Inventors: Sebastiano Banni, Cagliari (IT); Miriam Melis, Cagliari (IT); Marco Pistis, Cagliari (IT); Valeria Sogos, Cagliari (IT)

(73) Assignee: UNIVERSITA' DEGLI STUDI DI CAGLIARI, Cagliari (IT)

(21) Appl. No.: 15/329,968

(22) PCT Filed: Jul. 27, 2015
(86) PCT No.: PCT/IB2015/055664

§ 371 Date: Jan. 27, 2017

(30) Foreign Application Priority Data
Jul. 31, 2014 (IT) RM2014A000433

(51) Int. Cl. A61K 31/685; A61K 31/202; A61K 31/201; A61K 45/06; C11C 3/02

(52) U.S. Cl. CPC A61K 47/48053; A61K 31/201; A61K 31/202; A61K 45/06

(57) ABSTRACT

An ester of a phospholipid with conjugated linoleic acid for use in the therapeutic treatment of or as a food supplement for psychiatric disorders with neuroinflammatory and neurodegenerative basis, such as depression and schizophrenia.

The present invention relates to an ester of a phospholipid with conjugated linoleic acid for the treatment of psychiatric disorders with neuroinflammatory and neurodegenerative basis.

More in detail, the invention relates to an ester of a phospholipid with conjugated linoleic acid for use in therapeutic treatment of or as a food supplement for psychiatric disorders with neuroinflammatory and neurodegenerative basis, such as depression and schizophrenia.

Depression is bound to give the second largest contribution to the global load of diseases and disabilities within 2020 (World Health Organization), although it still represents a significant unmet clinical need. Studies that have employed modern techniques for evaluating depressive episodes in general population agree on the fact that 13-16% of adults have at least once in their life met the criteria for the Major Depressive Disorder (Kessler et al., 2003; Hasin et al., 2005), perhaps even reaching 20% when this datum is extrapolated by relating it to the whole length of life. In these samples of general population, the average depressive episode lasts 3 to 4 months (Spijker et al., 2002; Kessler et al., 2003; Eaton et al., 2008). About half the people who have suffered from it once then recovers and have no relapse (Eaton et al., 2008), even though approx. 20% of the depressive episodes have a chronic course lasting two years or longer (Spijker et al., 2002). These data reveal that depression cases in general population, about half of which are not diagnosed or treated (Kessler et al., 2003; Hasin et al., 2005; Eaton et al., 2008), are more common, have a shorter duration, and are often less recurring than emerges from studies conducted on clinical samples.

Schizophrenia (SZ) is a progressive neuropsychiatric disease that affects, in average, 1% of the population, and causes diffused compromission of behaviours and cognitive functions (Pantelis and Brewer, 1995). Cognitive deficits are present independently of the stage of development of the disease, and are particularly serious as concerns the tasks related to the operation of the frontal and temporal lobes, including attention, speed, executive functions, verbal memory and learning (Censits et al., 1997; Townsend et al., 2001). A certain number of neurobiological alterations have been associated with cognitive compromission, such as loss of white and gray matter and frontal lobe hypofunction (Weinberger et al., 1988; Price et al., 2010; de Castro-Manglano et al., 2011). Various molecular modifications have also been associated with the disease, including loss of synaptic proteins of the brain and increased neuroinflammation, excitotoxicity and arachidonic-acid metabolism markers (AA) (Chen et al., 2011; Rao et al., 2011b; Rao et al., 2011a; Rao et al., 2012). Similar modifications of these markers have also been found in dementia associated with HIV-1 (Everall et al., 1999; Aoki et al., 2005) and in bipolar disorder (Aoki et al., 2005; Kim et al., 2010).

In light of the above, it is apparent that there is a need for new methods of treatment of psychiatric disorders with neuroinflammatory and neurodegenerative basis, which can overcome the drawbacks of the therapies known in the art.

Some evidence suggests that depression is often triggered by stressful life events, that stress triggers neuronal microtraumatisms and neuroinflammatory activation in the brain, and that inflammatory mediators can induce depressive symptoms (Wager-Smith and Markou, 2011). Furthermore, it has recently being emerging that anti-inflammatory treatments have antidepressant effects, and that antidepressant therapies can improve, in addition to neurogenesis, also the effects of neurotrophines and neuronal plasticity (Wager-Smith and Markou, 2011).

Inflammation is an active defense reaction against different insults, which aims at neutralizing noxious agents. Although inflammation is useful as a protective function for controlling infections and promoting tissue repair, it may also cause damage to tissues (Laye, 2010). This mediated response of the brain leads, in particular, to profound metabolic alterations, in the form of a higher thermoregulatory set-point resulting in fever, and drastic behavioural changes commonly labelled as “diseased behaviour” (anorexy, locomotor activity reduction, social withdrawal, etc.). The expression of cytokines in the brain plays a key role in the physiopathology of immune neurological disorders (e.g. multiple sclerosis) and non-immune ones (e.g. brain damage, ictus, Alzheimer disease) (Laye, 2010).

In the brain, inflammatory mediators are mainly produced by endothelial and glial cells, including astrocytes and microglia (Rothwell and Luheshi, 2000). The expression of pro-inflammatory cytokines in the brain increases in response to various conditions, such as infections (bacteria, viruses), lesions, traumas and oxidative stress. Neuroinflammation, i.e. the inflammatory response in the brain, has many cellular and biochemical characteristics that make it different from peripheral inflammatory response.

Among the functional consequences of neuroinflammation we include cognitive and behavioural alterations, which usually occur in the absence of neurotoxicity (Dantzer, 2001). It is well known that the behavioural repertoire of human beings and animals changes drastically in the course of an infection. Diseased individuals are scarcely motivated to eat, are lazy, complain of tiredness and discomfort, lose interest in social activities, and exhibit significant sleep pattern changes. In addition, they feel sick and, being afflicted by pain, show inability to experience pleasure (anhedonia) and exhibit attention, concentration and memory difficulties (Dantzer et al., 2008). These alterations are responsible for a reduced quality of life and welfare, and can be reproduced in healthy individuals by means of peripheral or central injections of pro-inflammatory cytokines (Kent et al., 1996). When neuroinflammation is exacerbated or prolonged, it may lead to neuronal cellular death and neurodegeneration as a consequence of deprivation of neuronal growth factors or overproduction of reactive species of oxygen (Rothwell and Luheshi, 2000; Venters et al., 2000).

Neuroinflammation has many aspects, all of which occur simultaneously. Following exposition to harmful stimuli, some neuroinflammatory components include microglial activation, cytokine release, and induction of tissue repair enzymes, which together limit the cellular damage and promote the repair. At behavioural level, cytokine-induced diseased behaviour is nothing else than the exterior manifestation of a central motivational state that helps the organism fight the infection and promote the recovery (Dantzer, 2001). Pro-inflammatory cytokines act in the brain to induce non-specific infection symptoms, including fever and profound psychological and behavioural changes defined as “disease behaviour”. Affected subjects exhibit weakness, discomfort, cognitive alterations and laziness, hypersleep, depressed activity, and loss of interest in social activities (Dantzer, 2001). Although these symptoms are generally considered to be the result of the debilitating process occurring during the infection, they are actually part of a natural homeostatic reaction of the body for fighting against infections (Hart, 1988). These behavioural changes have been proved to be the expression of a motivational state that resets the priorities of the body to promote resistance to pathogens and recovery from the infection. Since it prevents metabolically costly activities and favours the expression of activities that promote calorie preservation (e.g. rest), diseased behaviour positively contributes to inflammation recovery (Dantzer, 2001).

There is much evidence in favour of a role played by cytokines in the mediation of mood disorders and cognitive disorders developing in patients treated with cytokines by immunotherapy (Capuron and Dantzer, 2003). The same mechanisms seem to operate for the wide variety of non-specific disease symptoms developing in patients suffering from somatic diseases characterized by an inflammatory component, including coronary disease, rheumatoid arthritis, asthma, cancer, ictus, and various neuropathologies (Eikelenboom et al., 2002; Gidron et al., 2002; Kiecolt-Glaser and Glaser, 2002; Cleeland et al., 2003). Many patients complain for pain, tiredness, anorexy, sleep disorders, and cognitive and mood disorders. These non-specific neurovegetative and psychiatric symptoms are not necessarily the result of a chain of events connected to each other by direct causality (e.g. pain causes sleep disorders, which have a negative impact on cognition and induce tiredness and exhaustion, leading to anorexy) (Cleeland et al., 2003). They may actually represent only another aspect of the inflammatory process. These non-specific symptoms are the main source of suffering for the patient, often more than the diseased organ.

Several studies have reported microglial activation (Steiner et al., 2008; van Berckel et al., 2008) and high levels of pro-inflammatory cytokines in post-mortem brain tissue of SZ patients, as well as high levels of cytokines in the plasma (Licinio et al., 1993; Drexhage et al., 2008; Muller and Schwarz, 2008). Microglial and astrocyte activation may induce, via the nuclear factor kappa B (NF-kB) pathway, release of pro-inflammatory cytokines (interleukine-1 beta (IL-1β)) and of the tumor necrosis factor alpha (TNFα), and also activation of numerous signal transduction pathways, including the AA cascade (Hernandez et al., 1999; Laflamme et al., 1999; Blais and Rivest, 2001; Moolwaney and Igwe, 2005). Chronic activation of the N-methyl-D-aspartate (NMDA) receptor causes selective over-regulation of mRNA, proteins, and activity of cytosolic phospholipase A2 (cPLA2) selective for AA in rat brain (Rao et al., 2007). During excitotoxic insults, specific excitotoxicity biomarkers, such as inducible nitric oxide synthase (iNOS) (Acarin et al., 2002) and c-Fos, are expressed in the brain (Rogers et al., 2005).

AA is mostly located in the position (sn)-2 of membrane phospholipids, from where it can be hydrolized by cPLA2 or by secretory sPLA2. The released AA can be converted, under the action of cyclooxygenase (COX), lipooxygenase (LOX) and thromboxane synthase (TXS) enzymes, into pro-inflammatory mediators, such as prostaglandins (PG) H2 and leukotrienes. The increase in AA brain markers has been reported in an experimental model of neuroinflammation in rats (Rosenberger et al., 2004; Basselin et al., 2011).

An association has been demonstrated, both in vivo and in vitro, between AA and its pro-inflammatory metabolytes and neuronal apoptosis and synapse loss (Okuda et al., 1994; Williams et al., 1998; Farooqui et al., 2001; Yagami et al., 2002; Fang et al., 2008). Also, the reduced density of dendritic spines and their complexity have been associated with deficits in learning, memory, and general cognitive functions (Masliah et al., 1997). In the SZ brain, associations between synapse loss, elevated AA cascade markers, neuroinflammation and loss of synaptic proteins have not yet been clearly identified as a pathogenetic characteristic of the disease. However, previous studies have reported structural, metabolic and neurotransmission anomalies in the frontal cortex of SZ patients (Weinberger et al., 1988; Beasley et al., 2009; Price et al., 2010; de Castro-Manglano et al., 2011).

Sustained neuroinflammation may also be one of the factors that trigger the development of depressive disorder and may be involved in the mediation of cellular apoptosis, which is the classic form of programmed cell death.

It has also been hypothesized that the progression of the disease and the cognitive deficits found in SZ may be associated with neuroinflammation and AA cascade activation; therefore, anti-neuroinflammatory therapies might be therapeutically useful.

An important anti-inflammatory role is played at brain level by peroxisomes via degradation of the pro-inflammatory eicosanoids derived from arachidonic acid by beta-oxidation (Diczfalusy et al., 1991; Diczfalusy et al., 1993; Ferdinandusse et al., 2002). Peroxisomial beta oxidation is regulated by a transcription factor, PPAR alpha, which exerts its action after binding with specific ligands (Rakhshandehroo et al., 2010). In addition, PPAR alpha activation plays an important role in the homeostasis of the dopaminergic system (Melis et al., 2010; Melis et al., 2013a; Melis et al., 2013b). Therefore, PPAR alpha might play a direct role in the modulation of the dopaminergic system, and might be the key factor allowing functional recovery of the dopaminergic system after an inflammatory event. On the contrary, non-activation of PPAR alpha might sustain inflammation and loss of homeostatic control of the dopaminergic system. The physiologic ligands of PPAR alpha are fatty acids which, depending on the length of the chain and on the number of double bonds, show different affinity towards this receptor. Therefore, the availability of different fatty acids regulates the activity of PPAR alpha. Eicosanoids derived from arachidonic acid, such as prostaglandins and leukotrienes, are endogenous ligands of PPAR alpha. A physiological mechanism can thus be assumed to exist, wherein the activation of PPAR alpha limits their concentration by increasing their catabolism. In addition, the PPAR alpha activation increases the biosynthesis of the amides of palmitic acid, i.e. palmitoylethanolamide (PEA), and of oleic acid, i.e. oleoylethanolamide (OEA) (Melis et al., 2013a), which in turn are ligands of PPAR alpha. High levels of PEA and OEA sustain PPAR alpha activation, thus ensuring anti-inflammatory activity and preservation of the homeostasis of the dopaminergic system. Therefore, in stressful and inflammatory-insult conditions, high cerebral levels of OEA and PEA might ensure an adequate anti-inflammatory response and neuronal functional preservation.

Conjugated linoleic acid (CLA) is a set of isomeric forms of linoleic acid, such as, for example, the 9c,11t and 10t,12c isomers. CLA is a fatty acid which is present at low concentrations in our diet, and which is an avid ligand of PPAR alpha (Moya-Camarena et al., 1999). CLA has several nutritional properties at peripheral level (Belury, 2002), but also great potentialities at the level of the central nervous system, since has been proved that it crosses the hematoencephalic barrier in both test animal (Fa et al., 2005) and man (Cappa et al., 2012).

Another very important factor that affects the nutritional activity of alimentary fatty acids is the form in which they are present. It has been demonstrated, in fact, that polyunsaturated fatty acids are more bioavailable in phospholipid form than in triglycerid form in many tissues (Batetta et al., 2009), including the brain (Di Marzo et al., 2010).

The inventors of the present invention have now found that conjugated linoleic acid, in particular the c9,t11 isomer, may be able to improve protection against neuroinflammation, induce a re-balance of the dopaminergic neuronal function, and enhance the synaptogenesis and neuritogenesis, thus turning out to be a very good alimentary support in psychiatric disorders with neuroinflammatory and neurodegenerative basis. This effect is much enhanced if CLA is esterified in phospolipids such as phosphatidylcholine, phosphatidylethanolamine, phosphatidylserine, since it is readily incorporated at the level of the central nervous system, where it performs its anti-inflammatory action through PPAR alpha activation and PEA and OEA increase.

Therefore, the results of the above-mentioned study demonstrate that CLA, and in particular the c9,t11 isomer, can increase the levels of PEA and OEA both ex vivo and in vivo, and hence sustain PPAR alpha activation. The PEA and OEA increase, since it sustains PPAR alpha activation, is crucial for the physiologic response to neuroinflammatory events, in addition to preserving the homeostasis of the dopaminergic system. In fact, CLA can reduce in vitro the levels of TNF alpha in both astrocytes and microglia.

The use of CLA in degenerative diseases which are characteristic of old age has been described in United States patent US20090169533, which shows experimental data concerning presbyacusis alone. More specifically, patent US20090169533 relates to the use of CLA as an individual component and/or mixed with alpha lipoic acid (ALA), Q10 and phenylalanine (PA) for prevention and treatment of neurodegenerative diseases, in particular those which are characteristic of old age, such as presbyacusis. The action mechanism hypothesized in this patent is assumed to be correlated to the antioxidant capability of ALA and Q10, and to the role played by PA as a precursor of L-tyrosine, and hence of dopamine, norepinephrine, epinephrine, of the neurotransmitters in the cochlea. On the other hand, CLA is assumed to act through its capability of reducing the body weight, similarly to caloric restriction, which has been proven to prevent presbyacusis (Someya et al. 2007). Therefore, the study described in patent US20090169533 appears to exclusively address neurodegenerative diseases correlated to old age, and is based on antioxidant capabilities and on the effect of caloric restriction for preventing the above-mentioned diseases.

The CLA activity demonstrated in the present invention is completely different from the one described in patent US20090169533. The present invention, in fact, describes a direct activity of CLA at the level of the central nervous system, which is disconnected from a peripheral activity, such as the one on adiposity, or from any activity correlated to neurodegeneration processes which are characteristic of old age. For this reason, the use of CLA in the form of phospholipidic ester is of fundamental importance to ensure the utmost bioavailability at brain level. Moreover, the preferred CLA isomer according to the present invention is c9,t11, which has no body weight reduction activity (Pariza et al. 2001), while the only CLA isomer capable of reducing body fat is t10,c12 (Pariza et al. 2001).

It is therefore a specific object of the present invention to provide an ester of a phospholipid, preferably phosphatidylcholine, phosphatidylethanolamine, phosphatidylserine, with conjugated linoleic acid or conjugated linoleic acid and docosahexaenoic acid (DHA), for use in the neuropsychiatric medical field. Therefore, the ester according to the present invention may consist of a phospholipid esterified with one or two molecules of conjugated linoleic acid or with one molecule of conjugated linoleic acid and one molecule of DHA.

Preferably, the conjugated linoleic acid is the c9,t11 isomer; according to the present invention one may use, therefore, either a mixture of isomers having a high concentration of said isomer, e.g. at least 80% of said isomer, or the pure isomer.

Esterification of the phospholipid with DHA as well, in addition to CLA, is advantageous because DHA also plays an important role for the brain function (Rapoport et al. 2011). It has been recently demonstrated that the DHA amide has an action that promotes the synaptogenesis and the neuritogenesis (Kim and Spector, 2013). Furthermore, DHA has also been found to be a high-affinity ligand of PPAR alpha (Diep et al., 2000). Therefore, administration of CLA and DHA simultaneously esterified as phospholipids allows them to be readily incorporated at the level of the central nervous system, where they perform their anti-inflammatory function via PPAR alpha activation and PEA and OEA increase, and via enhancement of neurogenesis by increasing the DHA amide.

It is a further object of the present invention to provide an ester of a phospholipid, such as, for example, phosphatidylcholine, phosphatidylethanolamine, phosphatidylserine, with conjugated linoleic acid or conjugated linoleic acid and docosahexaenoic acid (DHA) for use in treatment and prevention of psychiatric disorders, preferably having neuroinflammatory and neurodegenerative basis, including, without being limited to, psychotic disorders such as schizophrenia, schizophreniform disorder, schizoaffective disorder, or mood disorders such as major depression, dysthymic disorder.

Preferably, the conjugated linoleic acid is the c9,t11 isomer.

The present invention also relates to a pharmaceutical composition comprising or consisting of an ester of a phospholipid with conjugated linoleic acid or conjugated linoleic acid and docosahexaenoic acid as active ingredient, in association with one or more pharmaceutically acceptable excipients and/or adjuvants. As aforementioned, the phospholipid can be selected from, for example, phosphatidylcholine, phosphatidylethanolamine, phosphatidylserine. Preferably, the conjugated linoleic acid is the c9,t11 isomer.

The pharmaceutical composition of the invention can be used as a therapeutic composition or as a food supplement, and may further comprise antipsychotic and/or antidepressant drugs.

A further object of the present invention is the use of the pharmaceutical composition of the invention for treatment and prevention of psychiatric disorders preferably having a neuroinflammatory and neurodegenerative basis, such as, for example, psychotic disorders such as schizoprenia, schizophreniform disorder, schizoaffective disorder, or mood disorders such as major depression, dysthymic disorder.

The pharmaceutical composition of the invention may further comprise DHA.

A preferred embodiment of the present invention will now be described by way of non limiting example with particular reference to the annexed drawings, wherein:

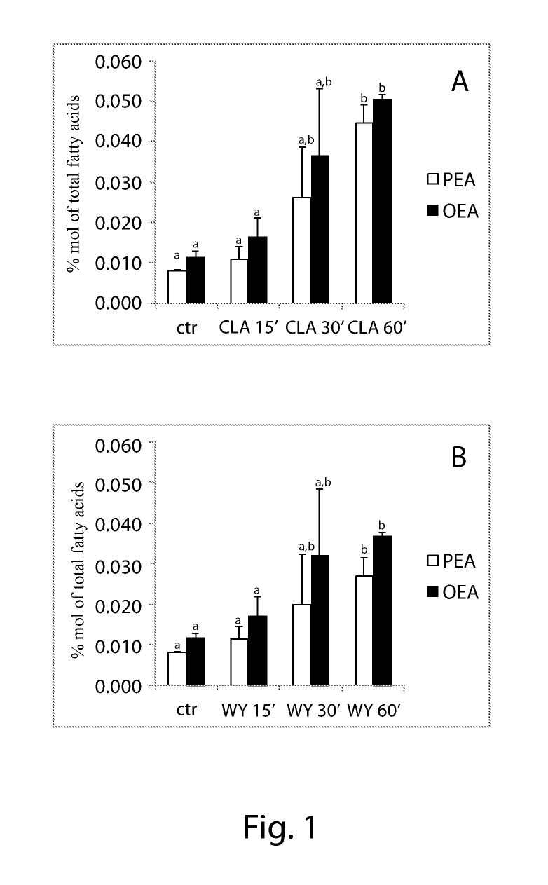

FIG. 1 shows the value of PEA and OEA in rat brain slices incubated with CLA (80% c9,t11 isomer) (A) or WY (B) for 15, 30 or 60 min. The values, mean and DS, are expressed as molar % of the total fatty acids. Different letters indicate significant differences (p<0.05); the One-way ANOVA statistical analysis was used, with Tukey test for post-hoc analysis. Similar results were obtained by using pure (99%) c9,t11 isomer.

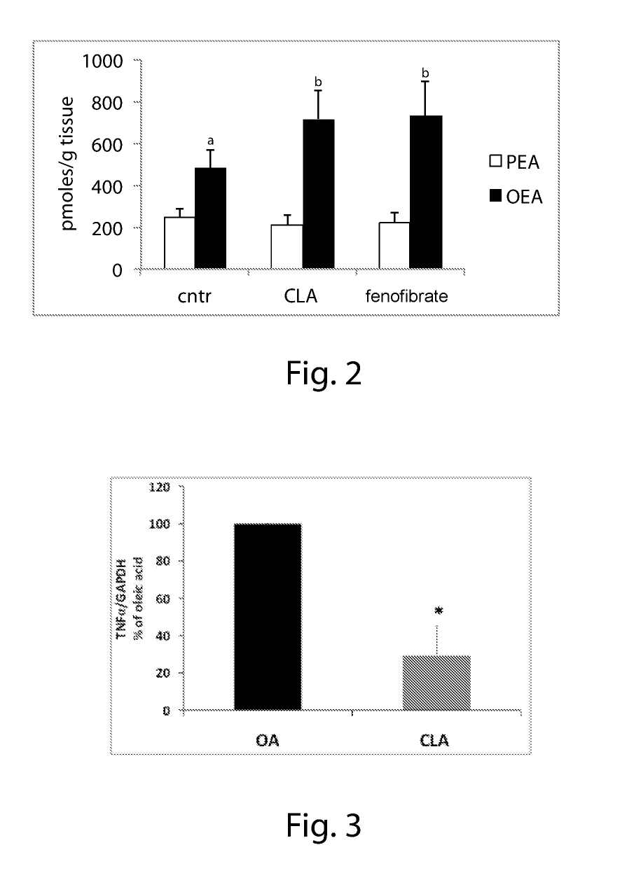

FIG. 2 shows the values of PEA and OEA in the frontal cortex of mice treated for 14 days with a diet containing 0.2% of fenofibrate or administration of CLA (80% c9,t11 isomer) 12 h prior to sacrifice. The values, mean and DS, are expressed as pmoles/g tissue. Different letters indicate significant differences (p<0.05); the One-way ANOVA statistical analysis was used, with Tukey test for post-hoc analysis.

FIG. 3 shows the values of TNF alpha in astrocytes after treatment with oleic acid (OA) or CLA (80% c9,t11 isomer) and subsequent treatment with LPS. The values, mean and DS, are expressed as % of the values obtained with oleic acid. * significantly different (p<0.05) with Student's t-test.

### EXAMPLE 1: EX VIVO AND IN VIVO STUDY ON THE EFFECT OF CLA ON THE LEVELS OF PEA, OEA AND TNF ALPHA

The methods employed for the analysis of the levels of conjugated linoleic acid, PEA and OEA in vivo and in vitro are described in detail in (Melis et al. 2001) and in (Piscitelli et al. 2011), respectively.

### Results

Studies conducted on brain slices have demonstrated how ex vivo incubation with CLA in free form, i.e. both with 80% c9,t11 isomer and with pure isomer, increases the levels of PEA and OEA (FIG. 1A) by about 5 times. On the contrary, WY14643 (WY), a synthetic agonist of PPAR alpha used as a comparison since it is an inductor of PPAR alpha, increases the levels of PEA and OEA by about 3-4 times (FIG. 1B).

In addition, acute in vivo administration of CLA (80% c9,t11 isomer), 12 hours prior to sacrifice and collection of brain sections, significantly increases the levels of OEA in the frontal cortex of mice, to a level comparable to that induced by a diet with fenofibrate, another known synthetic agonist of PPAR alpha (Puligheddu et al., 2013) (FIG. 2). Since astrocytes are involved in neuroinflammation, the action of CLA (80% c9,t11 isomer) was also verified in cultures of astrocytes after having induced neuroinflammation with LPS. Oleic acid was used as a control. The results clearly demonstrate a significant decrease of TNF alpha in cultured astrocytes (FIG. 3).

### BIBLIOGRAPHIC REFERENCES

Acarin L, Peluffo H, Gonzalez B, Castellano B (2002) Expression of inducible nitric oxide synthase and cyclooxygenase-2 after excitotoxic damage to the immature rat brain. Journal of neuroscience research 68:745-754.
Aoki C, Sekino Y, Hanamura K, Fujisawa S, Mahadomrongkul V, Ren Y, Shirao T (2005) Drebrin A is a postsynaptic protein that localizes in vivo to the submembranous surface of dendritic sites forming excitatory synapses. The Journal of comparative neurology 483:383-402.
Basselin M, Ramadan E, Chen M, Rapoport S I (2011) Anti-inflammatory effects of chronic aspirin on brain arachidonic acid metabolites. Neurochemical research 36:139-145.
Batetta B, Griinari M, Carta G, Murru E, Ligresti A, Cordeddu L, Giordano E, Sanna F, Bisogno T, Uda S, Collu M, Bruheim I, Di Marzo V, Banni S (2009) Endocannabinoids may mediate the ability of (n-3) fatty acids to reduce ectopic fat and inflammatory mediators in obese Zucker rats. Journal of Nutrition 139:1495-1501.
Beasley C L, Dwork A J, Rosoklij a G, Mann J J, Mancevski B, Jakovski Z, Davceva N, Tait A R, Straus S K, Honer W G (2009) Metabolic abnormalities in fronto-striatal-thalamic white matter tracts in schizophrenia. Schizophrenia research 109:159-166.
Belury M A (2002) Dietary conjugated linoleic acid in health: Physiological effects and mechanisms of action [Review]. Ann Rev Nutr 22:505-531.
Blais V, Rivest S (2001) Inhibitory action of nitric oxide on circulating tumor necrosis factor-induced NF-kappaB activity and COX-2 transcription in the endothelium of the brain capillaries. Journal of neuropathology and experimental neurology 60:893-905.
Cappa M, Bizzarri C, Petroni A, Carta G, Cordeddu L, Valeriani M, Vollono C, De Pasquale L, Blasevich M, Banni S (2012) A mixture of oleic, erucic and conjugated linoleic acids modulates cerebrospinal fluid inflammatory markers and improve somatosensorial evoked potential in X-linked adren.oleukodystrophy female carriers. J Inherit Metab Dis 35:899-907.
Capuron L, Dantzer R (2003) Cytokines and depression: the need for a new paradigm. Brain, behavior, and immunity 17 Suppl 1:S119-124.
Censits D M, Ragland J D, Gur R C, Gur R E (1997) Neuropsychological evidence supporting a neurodevelopmental model of schizophrenia: a longitudinal study. Schizophrenia research 24:289-298.
Chen K H, Reese E A, Kim H W, Rapoport S I, Rao J S (2011) Disturbed neurotransmitter transporter expression in Alzheimer's disease brain. Journal of Alzheimer's disease: JAD 26:755-766.
Cleeland C S, Bennett G J, Dantzer R, Dougherty P M, Dunn A J, Meyers C A, Miller A H, Payne R, Reuben J M, Wang X S, Lee B N (2003) Are the symptoms of cancer and cancer treatment due to a shared biologic mechanism? A cytokine-immunologic model of cancer symptoms. Cancer 97:2919-2925.
Dantzer R (2001) Cytokine-induced sickness behavior: mechanisms and implications. Annals of the New York Academy of Sciences 933:222-234.
Dantzer R, O'Connor J C, Freund G G, Johnson R W, Kelley K W (2008) From inflammation to sickness and depression: when the immune system subjugates the brain. Nature reviews Neuroscience 9:46-56.
de Castro-Manglano P, Mechelli A, Soutullo C, Gimenez-Amaya J, Ortuno F, McGuire P (2011) Longitudinal changes in brain structure following the first episode of psychosis. Psychiatry research 191:166-173.
Di Marzo V, Griinari M, Carta G, Murru E, Ligresti A, Cordeddu L, Giordano E, Bisogno T, Collu M, Batetta B, Uda S, Berge K, Banni S (2010) Dietary krill oil increases docosahexaenoic acid and reduces 2-arachidonoylglycerol but not N-acylethanolamine levels in the brain of obese Zucker rats. International Dairy Journal 20:231-235.
Diczfalusy U, Kase B F, Alexson S E, Bjorkhem I (1991) Metabolism of prostaglandin F2 alpha in Zellweger syndrome. Peroxisomal beta-oxidation is a major importance for in vivo degradation of prostaglandins in humans. The Journal of clinical investigation 88:978-984.
Diczfalusy U, Vesterqvist O, Kase B F, Lund E, Alexson S E (1993) Peroxisomal chain-shortening of thromboxane B2: evidence for impaired degradation of thromboxane B2 in Zellweger syndrome. J Lipid Res 34:1107-1113.
Diep Q N, Touyz R M, Schiffrin E L (2000) Docosahexaenoic acid, a peroxisome proliferator-activated receptor-alpha ligand, induces apoptosis in vascular smooth muscle cells by stimulation of p38 mitogen-activated protein kinase. Hypertension 36:851-855.
Drexhage R C, Padmos R C, de Wit H, Versnel M A, Hooijkaas H, van der Lely A J, van Beveren N, deRijk R H, Cohen D (2008) Patients with schizophrenia show raised serum levels of the pro-inflammatory chemokine CCL2: association with the metabolic syndrome in patients? Schizophrenia research 102:352-355.
Eaton W W, Shao H, Nestadt G, Lee H B, Bienvenu O J, Zandi P (2008) Population-based study of first onset and chronicity in major depressive disorder. Archives of general psychiatry 65:513-520.
Eikelenboom P, Bate C, Van Gool W A, Hoozemans J J, Rozemuller J M, Veerhuis R, Williams A (2002) Neuroinflammation in Alzheimer's disease and prion disease. Glia 40:232-239.
Esposito G, Giovacchini G, Liow J S, Bhattacharjee A K, Greenstein D, Schapiro M, Hallett M, Herscovitch P, Eckelman W C, Carson R E, Rapoport S I (2008) Imaging neuroinflammation in Alzheimer's disease with radiolabeled arachidonic acid and PET. Journal of nuclear medicine: official publication, Society of Nuclear Medicine 49:1414-1421.
Everall I P, Heaton R K, Marcotte T D, Ellis R J, McCutchan J A, Atkinson J H, Grant I, Mallory M, Masliah E (1999) Cortical synaptic density is reduced in mild to moderate human immunodeficiency virus neurocognitive disorder. HNRC Group. HIV Neurobehavioral Research Center. Brain pathology 9:209-217.
Fa M, Diana A, Carta G, Cordeddu L, Melis M P, Murru E, Sogos V, Banni S (2005) Incorporation and metabolism of c9,t11 and t10,c12 conjugated linoleic acid (CLA) isomers in rat brain. Biochim Biophys Acta 1736:61-66.
Fang K M, Chang W L, Wang S M, Su M J, Wu M L (2008) Arachidonic acid induces both Na+ and Ca2+ entry resulting in apoptosis. Journal of neurochemistry 104:1177-1189.
Farooqui A A, Yi Ong W, Lu X R, Halliwell B, Horrocks L A (2001) Neurochemical consequences of kainate-induced toxicity in brain: involvement of arachidonic acid release and prevention of toxicity by phospholipase A(2) inhibitors. Brain research Brain research reviews 38:61-78.
Ferdinandusse S, Meissner T, Wanders R J, Mayatepek E (2002) Identification of the peroxisomal beta-oxidation enzymes involved in the degradation of leukotrienes. Biochem Biophys Res Commun 293:269-273.
Gidron Y, Gilutz H, Berger R, Huleihel M (2002) Molecular and cellular interface between behavior and acute coronary syndromes. Cardiovascular research 56:15-21.
Harigaya Y, Shoji M, Shirao T, Hirai S (1996) Disappearance of actin-binding protein, drebrin, from hippocampal synapses in Alzheimer's disease. Journal of neuroscience research 43:87-92.
Hart B L (1988) Biological basis of the behavior of sick animals. Neurosci Biobehav Rev 12:123-137.
Hasin D S, Goodwin R D, Stinson F S, Grant B F (2005) Epidemiology of major depressive disorder: results from the National Epidemiologic Survey on Alcoholism and Related Conditions. Archives of general psychiatry 62:1097-1106.
Hatanpaa K, Isaacs K R, Shirao T, Brady D R, Rapoport S I (1999) Loss of proteins regulating synaptic plasticity in normal aging of the human brain and in Alzheimer disease. Journal of neuropathology and experimental neurology 58:637-643.
Hernandez M, Bayon Y, Sanchez Crespo M, Nieto M L (1999) Signaling mechanisms involved in the activation of arachidonic acid metabolism in human astrocytoma cells by tumor necrosis factor-alpha: phosphorylation of cytosolic phospholipase A2 and transactivation of cyclooxygenase-2. Journal of neurochemistry 73:1641-1649.
Huang S K, Klein D C, Korf H W (1992) Immunocytochemical demonstration of rod-opsin, S-antigen, and neuron-specific proteins in the human pineal gland. Cell and tissue research 267:493-498.
Kent S, Bret-Dibat J L, Kelley K W, Dantzer R (1996) Mechanisms of sickness-induced decreases in food-motivated behavior. Neurosci Biobehav Rev 20:171-175.
Kessler R C, Berglund P, Demler O, Jin R, Koretz D, Merikangas K R, Rush A J, Walters E E, Wang P S, National Comorbidity Survey R (2003) The epidemiology of major depressive disorder: results from the National Comorbidity Survey Replication (NCS-R). JAMA: the journal of the American Medical Association 289:3095-3105.
Kiecolt-Glaser J K, Glaser R (2002) Depression and immune function: central pathways to morbidity and mortality. Journal of psychosomatic research 53:873-876.
Kim H W, Rapoport S I, Rao J S (2010) Altered expression of apoptotic factors and synaptic markers in postmortem brain from bipolar disorder patients. Neurobiology of disease 37:596-603.
Kim H Y, Spector A A (2013) Synaptamide, endocannabinoid-like derivative of docosahexaenoic acid with cannabinoid-independent function. Prostaglandins Leukot Essent Fatty Acids 88:121-125.
Kojima N, Kato Y, Shirao T, Obata K (1988) Nucleotide sequences of two embryonic drebrins, developmentally regulated brain proteins, and developmental change in their mRNAs. Brain research 464:207-215.
Laflamme N, Lacroix S, Rivest S (1999) An essential role of interleukin-1beta in mediating NF-kappaB activity and COX-2 transcription in cells of the blood-brain barrier in response to a systemic and localized inflammation but not during endotoxemia. The Journal of neuroscience: the official journal of the Society for Neuroscience 19:10923-10930.
Layé S (2010) Polyunsaturated fatty acids, neuroinflammation and well being. Prostaglandins, leukotrienes, and essential fatty acids 82:295-303.
Licinio J, Seibyl J P, Altemus M, Charney D S, Krystal J H (1993) Elevated CSF levels of interleukin-2 in neuroleptic-free schizophrenic patients. The American journal of psychiatry 150:1408-1410.
Masliah E, Heaton R K, Marcotte T D, Ellis R J, Wiley C A, Mallory M, Achim C L, McCutchan J A, Nelson J A, Atkinson J H, Grant I (1997) Dendritic injury is a pathological substrate for human immunodeficiency virus-related cognitive disorders. HNRC Group. The HIV Neurobehavioral Research Center. Annals of neurology 42:963-972.
McCarthy M, Vidaurre I, Geffin R (2006) Maturing neurons are selectively sensitive to human immunodeficiency virus type 1 exposure in differentiating human neuroepithelial progenitor cell cultures. Journal of neurovirology 12:333-348.
Melis M P, Angioni E, Carta G, Murru E, Scanu P, Spada S, Banni S. Characterization of conjugated linoleic acid and its metabolites by RP-HPLC with diode array detector. Eur J Lipid Sci Technol 2001; 103(9):617-21.
Melis M, Carta G, Pistis M, Banni S (2013a) Physiological role of peroxisome proliferator-activated receptors type alpha on dopamine systems. CNS & neurological disorders drug targets 12:70-77.
Melis M, Carta S, Fattore L, Tolu S, Yasar S, Goldberg S R, Fratta W, Maskos U, Pistis M (2010) Peroxisome proliferator-activated receptors-alpha modulate dopamine cell activity through nicotinic receptors. Biol Psychiatry 68:256-264.
Melis M, Scheggi S, Carta G, Madeddu C, Lecca S, Luchicchi A, Cadeddu F, Frau R, Fattore L, Fadda P, Ennas M G, Castelli M P, Fratta W, Schilstrom B, Banni S, De Montis M G, Pistis M (2013b) PPARalpha regulates cholinergic-driven activity of midbrain dopamine neurons via a novel mechanism involving alpha7 nicotinic acetylcholine receptors. J Neurosci 33:6203-6211.
Moolwaney A S, Igwe O J (2005) Regulation of the cyclooxygenase-2 system by interleukin-1beta through mitogen-activated protein kinase signaling pathways: a comparative study of human neuroglioma and neuroblastoma cells. Brain research Molecular brain research 137:202-212.
Moya-Camarena S Y, Vanden Heuvel J P, Blanchard S G, Leesnitzer L A, Belury M A (1999) Conjugated linoleic acid is a potent naturally occurring ligand and activator of PPARalpha. J Lipid Res 40:1426-1433.
Muller N, Schwarz M J (2008) A psychoneuroimmunological perspective to Emil Kraepelins dichotomy: schizophrenia and major depression as inflammatory CNS disorders. European archives of psychiatry and clinical neuroscience 258 Suppl 2:97-106.
Okuda S, Saito H, Katsuki H (1994) Arachidonic acid: toxic and trophic effects on cultured hippocampal neurons. Neuroscience 63:691-699.
Pantelis C, Brewer W (1995) Neuropsychological and olfactory dysfunction in schizophrenia: relationship of frontal syndromes to syndromes of schizophrenia. Schizophrenia research 17:35-45.
Pariza M W, Park Y, Cook M E. The biologically active isomers of conjugated linoleic acid. Prog Lipid Res 2001; 40(4):283-98.
Piscitelli F, Carta G, Bisogno T, Murru E, Cordeddu L, Berge K, Tandy S, Cohn J S, Griinari M, Banni S, et al. Effect of dietary krill oil supplementation on the endocannabinoidome of metabolically relevant tissues from high-fat-fed mice. Nutr Metab 2011; 8:51.
Price G, Cercignani M, Chu E M, Barnes T R, Barker G J, Joyce E M, Ron M A (2010) Brain pathology in first-episode psychosis: magnetization transfer imaging provides additional information to M M measurements of volume loss. NeuroImage 49:185-192.
Puligheddu M, Pillolla G, Melis M, Lecca S, Marrosu F, De Montis M G, Scheggi S, Carta G, Murru E, Aroni S, Muntoni A L, Pistis M (2013) PPAR-Alpha Agonists as Novel Antiepileptic Drugs: Preclinical Findings. PLoS One 8:e64541.
Rakhshandehroo M, Knoch B, Muller M, Kersten S (2010) Peroxisome Proliferator-Activated Receptor Alpha Target Genes. PPAR research 2010:Article ID 612089.
Rao J S, Rapoport S I, Kim H W (2011a) Altered neuroinflammatory, arachidonic acid cascade and synaptic markers in postmortem Alzheimer's disease brain. Translational psychiatry 1:e31.
Rao J S, Ertley R N, Rapoport S I, Bazinet R P, Lee H J (2007) Chronic NMDA administration to rats up-regulates frontal cortex cytosolic phospholipase A2 and its transcription factor, activator protein-2. Journal of neurochemistry 102:1918-1927.
Rao J S, Kellom M, Kim H W, Rapoport S I, Reese E A (2012) Neuroinflammation and synaptic loss. Neurochemical research 37:903-910.
Rao J S, Kim H W, Kellom M, Greenstein D, Chen M, Kraft A D, Harry G J, Rapoport S I, Basselin M (2011b) Increased neuroinflammatory and arachidonic acid cascade markers, and reduced synaptic proteins, in brain of HIV-1 transgenic rats. Journal of neuroinflammation 8:101.
Rapoport S I, Ramadan E, Basselin M. Docosahexaenoic acid (DHA) incorporation into the brain from plasma, as an in vivo biomarker of brain DHA metabolism and neurotransmission. Prostaglandins Other Lipid Mediat 2011; 96(1-4):109-13.
Rogers A, Schmuck G, Scholz G, Griffiths R, Meredith C, Schousboe A, Campiani G, Williams D C (2005) Improvements in an in-vitro assay for excitotoxicity by measurement of early gene (c-fos mRNA) levels. Archives of toxicology 79:129-139.
Rosenberger T A, Villacreses N E, Hovda J T, Bosetti F, Weerasinghe G, Wine R N, Harry G J, Rapoport S I (2004) Rat brain arachidonic acid metabolism is increased by a 6-day intracerebral ventricular infusion of bacterial lipopolysaccharide. Journal of neurochemistry 88:1168-1178.
Rothwell N J, Luheshi G N (2000) Interleukin 1 in the brain: biology, pathology and therapeutic target. Trends in neurosciences 23:618-625.
Someya S, Yamasoba T, Weindruch R, Prolla T A, Tanokura M (2007) Caloric restriction suppresses apoptotic cell death in the mammalian cochlea and leads to prevention of presbycusis. Neurobiol Aging 28:1613-1622.
Spijker J, de Graaf R, Bijl R V, Beekman A T, Ormel J, Nolen W A (2002) Duration of major depressive episodes in the general population: results from The Netherlands Mental Health Survey and Incidence Study (NEMESIS). The British journal of psychiatry: the journal of mental science 181:208-213.
Steiner J, Bielau H, Brisch R, Danos P, Ullrich O, Mawrin C, Bernstein H G, Bogerts B (2008) Immunological aspects in the neurobiology of suicide: elevated microglial density in schizophrenia and depression is associated with suicide. Journal of psychiatric research 42:151-157.
Terry-Lorenzo R T, Inoue M, Connor J H, Haystead T A, Armbruster B N, Gupta R P, Oliver C J, Shenolikar S (2000) Neurofilament-L is a protein phosphatase-1-binding protein associated with neuronal plasma membrane and post-synaptic density. The Journal of biological chemistry 275:2439-2446.
Townsend L A, Malla A K, Norman R M (2001) Cognitive functioning in stabilized first-episode psychosis patients. Psychiatry research 104:119-131.
van Berckel B N, Bossong M G, Boellaard R, Kloet R, Schuitemaker A, Caspers E, Luurtsema G, Windhorst A D, Cahn W, Lammertsma A A, Kahn R S (2008) Microglia activation in recent-onset schizophrenia: a quantitative (R)-[11C]PK11195 positron emission tomography study. Biological psychiatry 64:820-822.
Venters H D, Dantzer R, Kelley K W (2000) A new concept in neurodegeneration: TNFalpha is a silencer of survival signals. Trends in neurosciences 23:175-180.
Wager-Smith K, Markou A (2011) Depression: a repair response to stress-induced neuronal microdamage that can grade into a chronic neuroinflammatory condition? Neuroscience and biobehavioral reviews 35:742-764.
Weinberger D R, Berman K F, Illowsky B P (1988) Physiological dysfunction of dorsolateral prefrontal cortex in schizophrenia. III. A new cohort and evidence for a monoaminergic mechanism. Archives of general psychiatry 45:609-615.
Williams J R, Leaver H A, Ironside J W, Miller E P, Whittle I R, Gregor A (1998) Apoptosis in human primary brain tumours: actions of arachidonic acid. Prostaglandins Leukot Essent Fatty Acids 58:193-200.
World Health Organization Dhwwimhmdde In.
Yagami T, Ueda K, Asakura K, Hata S, Kuroda T, Sakaeda T, Takasu N, Tanaka K, Gemba T, Hori Y (2002) Human group IIA secretory phospholipase A2 induces neuronal cell death via apoptosis. Molecular pharmacology 61:114-126.

1. An ester of a phospholipid with conjugated linoleic acid or conjugated linoleic acid and docosahexaenoic acid for use in the treatment of psychiatric disorders.

2. The ester according to claim 1, for use in the treatment of psychiatric disorders with neuroinflammatory and neurodegenerative basis.

3. The ester according to claim 1, for use in the treatment and the prevention of psychiatric disorders, preferably having neuroinflammatory and neurodegenerative basis.

4. The ester according to claim 1, wherein said phospholipid is selected from the group consisting of phosphatidylcholine, phosphatidylethanolamine, phosphatidylserine.

5. The ester according to claim 1, wherein the conjugated linoleic acid is the c9,t11 isomer.

6. The ester according to claim 1, wherein the psychiatric disorders with neuroinflammatory and neurodegenerative basis are selected from the group consisting of psychotic disorders such as schizophrenia, schizophreniform disorder, schizoaffective disorder, or mood disorders such as major depression, dysthymic disorder.

7. A pharmaceutical composition comprising of an ester of a phospholipid with conjugated linoleic acid or conjugated linoleic acid and docosahexaenoic acid as active ingredient, in association with one or more pharmaceutically acceptable excipients and/or adjuvants, for use in the treatment and the prevention of psychiatric disorders.

8. The pharmaceutical composition according to claim 7, wherein said phospholipid is selected from the group consisting of phosphatidylcholine, phosphatidylethanolamine, phosphatidylserine.

9. The pharmaceutical composition according to claim 7, wherein the conjugated linoleic acid is the c9,t11 isomer.

10. The pharmaceutical composition according to claim 7, further comprising antipsychotic and/or antidepressant drugs.

11. The pharmaceutical composition according to claim 7, wherein said pharmaceutical composition is a therapeutic composition or a food supplement.

12. The pharmaceutical composition as defined in claim 7, for use in the treatment and the prevention of psychiatric disorders having neuroinflammatory and neurodegenerative basis.

13. The pharmaceutical composition according to claim 12, wherein the psychiatric disorders with neuroinflammatory and neurodegenerative basis are selected from the group consisting of psychotic disorders such as schizophrenia, schizophreniform disorder, schizoaffective disorder, or mood disorders such as major depression, dysthymic disorder.
